(19) United States
(12) Patent Application Publication
(10) Pub. No.: US 2022/0192992 A1
(43) Pub. Date: Jun. 23, 2022

(54) PROTECTION OF MONOCLONAL ANTIBODY INTEGRITY AGAINST EVAPORATIVE SOLIDIFICATION, COMPRESSION AND PROTEOLYSIS BY DEXTRAN AND CYCLODEXTRIN DERIVATIVES

(71) Applicant: VTA Labs, LLC, Louisville, KY (US)

(72) Inventors: George A. Digenis, Louisville, KY (US); Mahendra K. Sreeramoju, Louisville, KY (US); Ahmad Malkawi, Louisville, KY (US)

(21) Appl. No.: 17/598,962

(22) PCT Filed: Apr. 1, 2020
(86) PCT No.: PCT/US2020/026092

§ 371 Date: Sep. 28, 2021

Related U.S. Application Data
(60) Provisional application No. 62/854,454, filed on May 30, 2019.
(60) Provisional application No. 62/827,419, filed on Apr. 1, 2019.

(51) Int. Cl. A61K 9/20; A61K 31/724; C07K 16/18

(52) U.S. Cl. CPC A61K 9/2095; C07K 16/18; A61K 31/724

(57) ABSTRACT

A powder comprises one or more monoclonal antibody, one or more cyclodextrin, and a compound selected from carboxymethyl dextran (CMD), one or more basic amino acid, or both. The powder may be compressed to form a compressed shape such as minitabs. A method of forming a powder comprises the steps of: 1) providing one or more monoclonal antibody, one or more cyclodextrin, and a compound selected from carboxymethyl dextran (CMD), one or more basic amino acid, or both; 2) forming a solution comprising the monoclonal antibody, cyclodextrin, CMD, and amino acid; and 3) drying the solution. This complexed mAb was found to be stable in resisting aggregation during the process of evaporative solidification and compression at pressures up to 10.5 kbar. Also, the complexed mAb has more resistance towards proteolysis than that of uncomplexed mAb.

### CROSS REFERENCE TO RELATED APPLICATIONS

The present application hereby claims the benefit of the provisional patent application of the same title, Ser. No. 62/827,419, filed on Apr. 4, 2019, and the provisional application titled, “Design of a Single Delivery System Containing a Monoclonal Antibody for the Simultaneous Treatment of Crohn's Disease and Ulcerative Colitis”, Ser. No. 62/854,454, filed on May 30, 2019, the disclosures of which are herein incorporated by reference in their entirety.

### BACKGROUND

Aggregation is a phenomenon where proteins/antibodies physically associate to yield large chemical entities with undesirable therapeutic effects. As a consequence, aggregation is a major challenge in the development of stable pharmaceutical formulations, particularly in manufacturing processes of therapeutic proteins and antibodies. Specifically, in the development of oral formulation of antibodies (in tablet form), it is essential to overcome aggregation and aggregate growth upon compression where higher pressures are applied (Truong-Le, V.; Lovalenti, P. M.; Abdul-Fattah, A. M., Stabilization Challenges and Formulation Strategies Associated with Oral Biologic Drug Delivery Systems. Advanced Drug Delivery Reviews 2015, 93, 95-108.). Thus, during the formulation development of tablets/minitabs, it is important to introduce methods or design formulations to preserve the integrity of antibody by restricting aggregation.

Although proteolysis by digestive enzymes such as pepsin and pancreatin is a well-known phenomenon (Asselin, J.; Hebert, J.; Amiot, J., Effects of In Vitro Proteolysis on the Allergenicity of Major Whey Proteins. Journal of Food Science 1989, 54 (4), 1037-1039), a major challenge in the oral formulation of mAbs is the protection of these molecules (peptides, proteins and antibodies) against various digestive enzymes such as pepsin (in the gastric segment of the GI tract) and pancreatin (in the intestinal segment of the GI tract) (Mitragotri, S.; Burke, P. A.; Langer, R., Overcoming the challenges in administering biopharmaceuticals: formulation and delivery strategies. Nature Reviews Drug Discovery 2014, 13, 655. Reilly, R. M.; Domingo, R.; Sandhu, J., Oral Delivery of Antibodies. Clinical Pharmacokinetics 1997, 32 (4), 313-323.). These enzymes are known to hydrolyze proteins, peptides and antibodies forming degradation products that have undesirable therapeutic effects. Accordingly, there is a need for methods to chemically stabilize and protect biomolecules against proteolysis, allowing these biomolecules to deliver their therapeutic effects when administered orally.

### BRIEF SUMMARY

A powder comprises one or more monoclonal antibody, one or more cyclodextrin, and a compound selected from carboxymethyl dextran (CMD), one or more basic amino acid, or both. The powder may be compressed to form a compressed shape such as minitabs.

A method of forming a powder comprises the steps of: 1) providing one or more monoclonal antibody, one or more cyclodextrin, and a compound selected from carboxymethyl dextran (CMD), one or more basic amino acid, or both; 2) forming a solution comprising the monoclonal antibody, cyclodextrin, CMD, and amino acid; and 3) drying the solution.

These and other objects and advantages shall be made apparent from the accompanying drawings and the description thereof.

### BRIEF DESCRIPTION OF THE FIGURES

The accompanying drawings, which are incorporated in and constitute a part of this specification, illustrate embodiments, and together with the general description given above, and the detailed description of the embodiments given below, serve to explain the principles of the present disclosure.

FIG. 1 is Schematic representation of various stages involved in producing mAb minitabs drug product including evaporative solidification, excipients blending, and minitabs compression.

FIG. 2 is a graph of percent aggregates in various mAb samples complexed with various proportions of CMD and HPBCD before and after evaporative solidification.

FIG. 3 is a graph of percent aggregates in mAb samples complexed with various proportions of CMD and HPBCD before and after evaporative solidification and minitabs compression at various compression pressures.

FIG. 4 is a graph of percent aggregates in antibodies (other than VTA-17) during solidification and compression. TRA is trastuzumab, ADA is adalimumab and BEV is bevacizumab

FIG. 5 is a graph of percent aggregates in mAb samples complexed with HPBCD, arginine and histidine before and after evaporative solidification and minitabs compression.

FIG. 6 is a graph of percent of remaining mAb formulated with and without CMD & HPBCD after incubation with simulated pancreatic juice (0.9 mg/ml pancreatin) for 5 hours at 37° C.

### DETAILED DESCRIPTION

In an attempt to convert a liquid formulation of a monoclonal antibody (mAb) into a dry powder form by a spray drying process, it was unexpectedly discovered that the presence of one or more cyclodextrin, and a compound selected from carboxymethyl dextran (CMD), one or more basic amino acid, or both, substantially improves the mAb's resistance to aggregation during evaporative solidification and subsequent compression into 2 mm diameter minitabs. In addition, CMD and HPBCD increase the resistance of the mAb to proteolytic destruction by the digestive enzyme pancreatin. The unusual protection properties of CMD, HPBCD, and basic amino acids greatly contribute to the successful development of an oral solid drug product of the mAb.

Complexation of mAb with stabilizers (such as HPBCD, CMD and basic amino acids), followed by evaporative solidification resulted in mAb powders with a desirable fluidity necessary for manufacturing of minitabs with active mAb. This complexed mAb was found to be stable in resisting aggregation during the process of evaporative solidification and compression at pressures up to 10.5 kbar. Also, the complexed mAb has more resistance towards proteolysis than that of uncomplexed mAb. The mAb of complexed powders has survived more than 45% after 2 hours of incubation in intestinal fluids with pancreatin concentration of 0.9 mg/mL that simulates fed conditions. The 45% survival of mAb in such a strong proteolytic milieu is found to be much better than what literature indicates (Truong-Le, V.; Lovalenti, P. M.; Abdul-Fattah, A. M., Stabilization Challenges and Formulation Strategies Associated with Oral Biologic Drug Delivery Systems. Advanced Drug Delivery Reviews 2015, 93, 95-108).

Aggregation (which results often in denaturation) of proteins as a function of pressure is a commonly encountered problem. Back in 1914, it was reported that a pressure of 7 kbar was able to denature proteins of egg white (Mozhaev, V. V.; Heremans, K.; Frank, J.; Masson, P.; Balny, C., High pressure effects on protein structure and function. Proteins: Structure, Function, and Bioinformatics 1996, 24 (1), 81-91. Bridgman, P. W., The Coagulation of Albumin by Pressure. Journal of Biological Chemistry 1914, 19, 511-512.). Application of high pressure induces either local or global changes in the protein structure and finally may lead to denaturation. While the pressure of 1-2 kbar is sufficient to cause dissociation of oligomeric and multiprotein complexes, denaturation of monomeric proteins is induced at a pressure range of 4-8 kbar (J L Silva, a.; Weber, G., Pressure Stability of Proteins. Annual Review of Physical Chemistry 1993, 44 (1), 89-113. Heremans, K., High Pressure Effects on Proteins and other Biomolecules. Annual Review of Biophysics and Bioengineering 1982, 11 (1), 1-21.).

In some embodiments, a powder comprises one or more monoclonal antibody, one or more cyclodextrin, and a compound selected from carboxymethyl dextran (CMD), one or more basic amino acid, or both. In some embodiments, the powder comprises about 20% to about 40% of one or more monoclonal antibody, about 35% to about 70% of one or more cyclodextrin, and about 35% to about 70% CMD. In some embodiments, the powder comprises about 20% to about 40% of one or more monoclonal antibody, about 35% to about 70% HPBCD, and about 35% to about 70% CMD. In some embodiments, the powder comprises about 20% to about 40% of one or more monoclonal antibody, about 45% to about 70% of one or more cyclodextrin, and about 15% to about 25% of one or more basic amino acid. In some embodiments, the powder comprises about 20% to about 40% of one or more monoclonal antibody, about 45% to about 70% HPBCD, and about 15% to about 25% of one or more basic amino acid.

In some embodiments, a powder comprises one or more monoclonal antibody, one or more cyclodextrin, carboxymethyl dextran (CMD), and one or more basic amino acid.

Cyclodextrins are a class of oligosaccharide macromolecules with a shape of a hollow truncated structure with hydrophilic exterior and hydrophobic interior. Examples of cyclodextrins include, but are not limited to: 2-hydroxy propyl beta cyclodextrin (HPBCD) and sulfobutylether beta cyclodextrin (SBECD). In some embodiments, the cyclodextrin is 2-hydroxy propyl beta cyclodextrin (HPBCD). In some embodiments, the cyclodextrin is sulfobutylether beta cyclodextrin (SBECD).

Carboxymethyl dextran (CMD) is a linear polymer with a (1-6)-linked glucose chains with low percentage (2-5%) of a (1-3) branches. CMDs are polyanionic in character due to the presence of about 5% negatively charged carboxyl groups (Gekko, K.; Noguchi, H., Selective interaction of calcium and magnesium ions with ionic dextran derivatives. Carbohydrate Research 1979, 69 (1), 323-326.). The molecular weights for the CMD range from about 40 kDa to about 500 kDa.

Examples of monoclonal antibodies include, but are not limited to VTA-17, trastuzumab, adalimumab, bevacizumab, or combinations thereof. In some embodiments, the monoclonal antibody is VTA-17.

Examples of basic amino acids include, but are not limited to arginine, histidine, and lysine. In some embodiments, the one or more basic amino acid comprises an amino acid selected from arginine, histidine, or both. In some embodiments, the basic amino acids are their acid salts.

The procedure for incorporation of monoclonal antibodies (mAbs) into a formulation consisting of 2 mm-diameter minitabs that can eventually be filled into hard gelatin capsules, is described below.

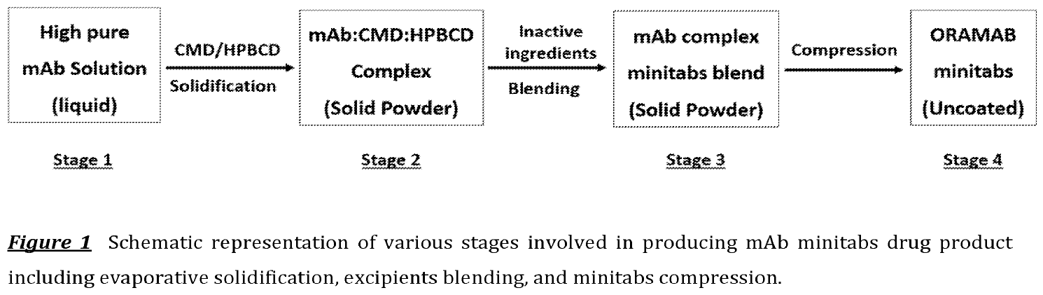

To attain desirable hardness with the minitabs, compression pressure up to 10.5 kbar is applied. The minitab compression involves two major processes, namely, the drying of the mAb solution to a dry powder state with desirable fluidity followed by compression into 2-3 mm minitabs. Examples of solidification processes include complexation of mAb, such as, VTA-17, with inactive ingredients such as carboxymethyl dextran (CMD), 2-hydroxypropyl β-cyclodextrin (HPBCD), arginine HCl, and histidine HCl, at various proportions. The solution is vacuum evaporation under controlled temperature and pressure. The compression process includes blending the mAb powder with inactive ingredients, such as binders, glidants, and lubricants. The various stages involved in the production of compressed minitabs are shown in FIG. 1.

In some embodiments, the active ingredient is a monoclonal antibody (mAb) called VTA-17, which is an anti-tumor necrosis factor alpha (TNFα) monoclonal antibody (mAb) obtained from the milk of transgenic goats (U.S. Pat. No. 7,939,317). This anti-TNFα antibody (mAb) is a glycoprotein in which its protein portion (aglycon) has the sequence of amino acids identical to that of adalimumab. However, it differs from adalimumab in its polysaccharide (glycon) portion. The mAb is obtained in solid form from an acetate buffer solution by evaporation under controlled temperature and pressure. This drying method developed for this invention is called “evaporative solidification” and produces the mAb material in a powder form having adequate fluidity that permits its compression into minitabs (with a diameter in the range of 2.0 to 3.0 mm) in a production scale without alteration of its potency.

It has been found that the mAb, VTA-17, is protected against aggregation by the use of compounds such as carboxymethyl dextran (CMD), 2-hydroxypropyl β-cyclodextrin (HPBCD), arginine, and histidine, during the evaporative solidification and compression. Also, upon complexation with HPBCD & CMD using the present method, other monoclonal antibodies like adalimumab, bevacizumab, and transtuzumab, were also found to resist the aggregation during evaporative solidification and compression. In addition, the CMD-HPBCD complexation was found to increase the resistance of the VTA-17 to proteolysis by digestive enzymes such as pancreatin.

It is desirable for powder blends containing a mAb that are compressed into minitabs that the integrity of mAb is maintained so the activity is not reduced. Desirable flow properties also make it easier to manufacture the minitabs in large production scale. Previously, VTA-17 solid was obtained from spray-drying a solution of VTA-17 in acetate buffer. Both CMD and HPBCD were used as supporting materials for the VTA-17 during the spray-drying (WO 2018/019900). However, the spray-dried mAb material was found to be sticky and not suitable for compression. Several approaches to improve the fluidity properties of this material failed. It was discovered that, a unique method of redisolving spray-dried material in water or phosphate buffer and solidification through slow evaporation under controlled temperature and reduced pressure transformed the material into a flowable powder. The spray-drying method was omitted and this unique method of solidification named as ‘evaporative solidification’ using a rotary-evaporator was applied directly to a solution of the mAb in acetate buffer following filtration to obtain a dried solid form of VTA-17.

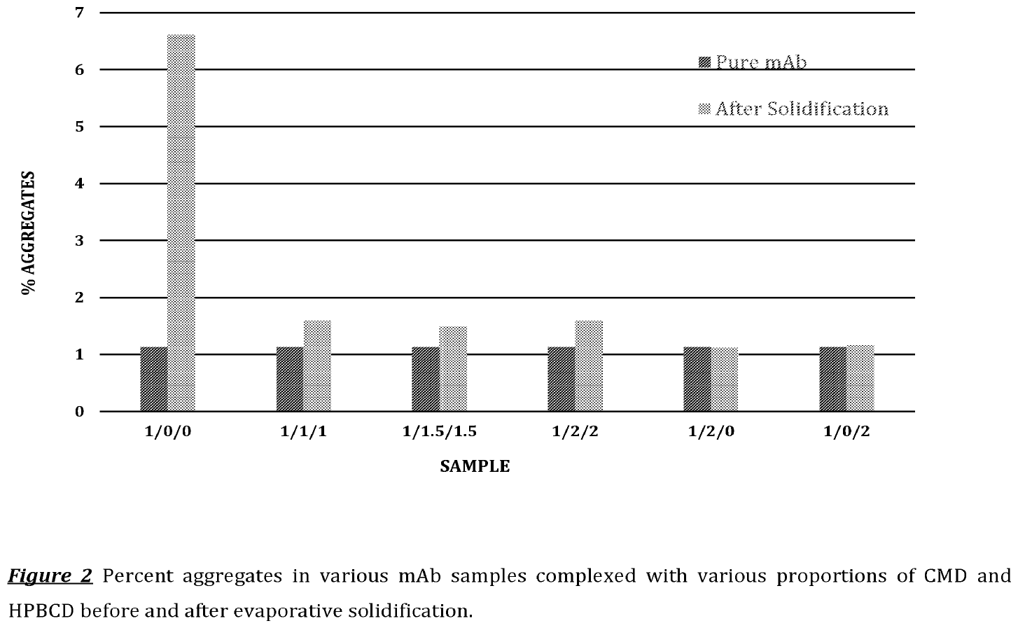

The process of evaporative solidification involves application of heating, continuous rotation, and evaporation under reduced pressure. It is required to ascertain that the integrity of mAb is preserved during this process by controlling the growth of aggregates and degradation products. During this process of evaporative solidification, the aggregates grew to about 6.6%, which is considered a significant improvement in the integrity of the mAb. CMD and HPBCD surprisingly diminish the aggregate growth significantly during evaporative solidification. The mAb that is complexed with either of CMD & HPBCD or both at various proportions showed very little to no growth in aggregates after evaporative solidification. The results are shown in FIG. 2 and Table 2. It is clear that these two excepients individually or together do prevent the aggregate growth during evaporative solidification, a process which is necessary to produce VTA-17 solid powder with desirable fluidity properties. More surprisingly, these excipients perform better individually than together (see 1/2/0 & 1/0/2 samples in FIG. 2 & Table 2). The aggregate growth in the presence of a mixture of CMD and HPBCD individually is almost nonexistent whereas in the presence of CMD and HPBCD together, it is about 0.5% (FIG. 2 & Table 2).

TABLE 2
Percent aggregates in various mAb samples complexed with various
proportions of CMD and HPBCD before and after evaporative
solidification, a process to obtain mAb with appropriate fluidity properties.

| Sample | 1/0/0 | 1/1/1 | 1/1.5/1.5 | 1/2/2 | 1/2/0 | 1/0/2 |
|---|---|---|---|---|---|---|
| Pure mAb | 1.13 | 1.13 | 1.13 | 1.13 | 1.13 | 1.13 |
| After Solidification | 6.61 | 1.59 | 1.49 | 1.59 | 1.12 | 1.16 |

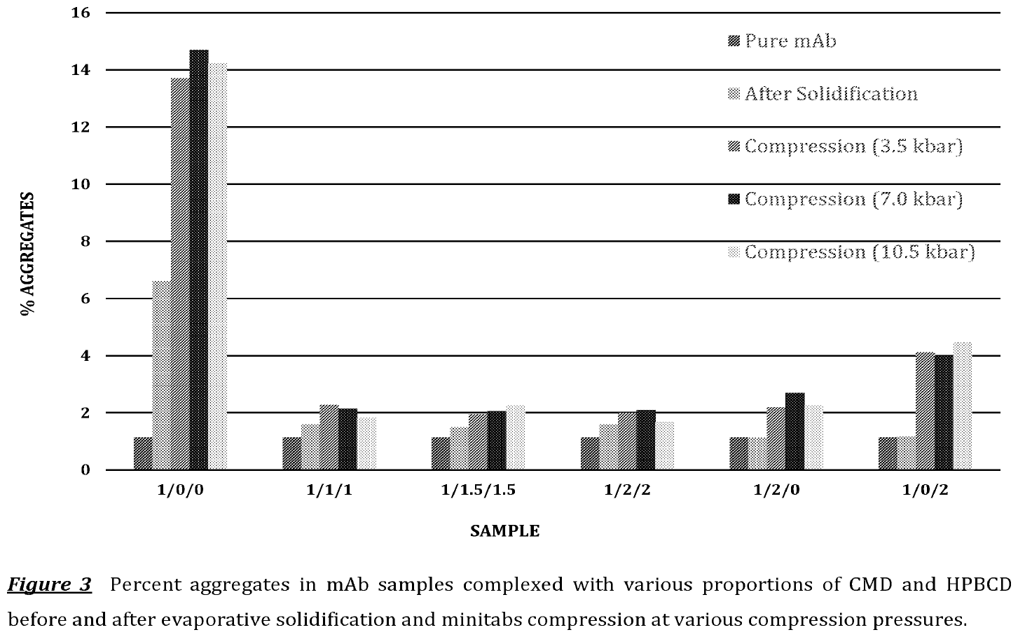

It was discovered that CMD & HPBCD individually and together protect VTA-17 from aggregation not only during evaporative solidification but also during minitabs compression. Since VTA-17 is intended to be incorporated into minitabs, it is necessary that the integrity is protected during tablet pressing. Minitabs were compressed at pressures in the range of 3.5-10.5 kbar, which is significantly higher than typical denaturing pressure of proteins. Upon compression, the percent aggregation of uncomplexed VTA-17 grew to around 15% (see sample 1/0/0 in FIG. 3 & Table 3) whereas for the mAb complexed with CMD and HPBCD, the percent aggregation is minimal, around 2% (FIG. 3 & Table 3). We checked the influence of these excipients separately during the compression and surprisingly found that CMD by itself is equally efficient (see sample 1/2/0 in FIG. 3 & Table 3) as CMD & HPBCD together, which are more efficient than HPBCD by itself. After the compression of uncomplexed mAb (1/0/0), the percent aggregation is 14.2% at 10.5 kbar (see sample 1/0/0 in FIG. 3 & Table 3 at 10.5 kbar pressure), which is the highest of the pressures applied. For all the mAb samples complexed with both CMD & HPBCD (1/1/1, 1/1.5/1.5 & 1/2/2) by weight, the percent of aggregation is well controlled to around 2%. For mAb complexed with exclusively HPBCD (see sample 1/0/2 in FIG. 3 & Table 3), the percent of aggregation is around 4.5%, higher than that of CMD and HPBCD together. But for mAb complexed with exclusively CMD, the percent of aggregation is well controlled around 2% like that of CMD and HPBCD together.

TABLE 3
Percent aggregates in various mAb samples complexed with various
proportions of CMD and HPBCD before and after evaporative
solidification and minitabs compression at various compression pressures

| Sample | | 1/0/0 | 1/1/1 | 1/1.5/1.5 | 1/2/2 | 1/2/0 | 1/0/2 |
|---|---|---|---|---|---|---|---|
| Pure mAb | | 1.13 | 1.13 | 1.13 | 1.13 | 1.13 | 1.13 |
| After Solidification | | 6.61 | 1.59 | 1.49 | 1.59 | 1.12 | 1.16 |
| Compression | 3.5 | 13.7 | 2.28 | 1.95 | 1.99 | 2.19 | 4.11 |
| (kbar) | 7.0 | 14.7 | 2.15 | 2.05 | 2.1 | 2.7 | 3.97 |
| | 10.5 | 14.2 | 1.83 | 2.25 | 1.68 | 2.25 | 4.47 |

Cyclodextrins are a class of oligosaccharide macromolecules with a shape of a hollow truncated structure with hydrophilic exterior and hydrophobic interior. Carboxymethyl dextran (CMD) on the other hand is a linear polymer with a (1-6)-linked glucose chains with low percentage (2-5%) of a (1-3) branches. CMDs are polyanionic in character due to the presence of about 5% negatively charged carboxyl groups. CMDs are assumed to make non-covalent and/or electrostatic interactions with protein backbone entities, thereby inducing a charged environment around the protein in solution. With these unique set of properties, both CMD and HPBCD were shown to protect VTA-17 against aggregation during evaporative solidification and compression. Especially during compression, CMD is playing a better role than HPBCD in resisting the aggregation induced during the process. Indeed, the preservation of the integrity of the mAb undergoing evaporative solidification and compression was further established by an ELISA assay proving no change in potency of the mAb.

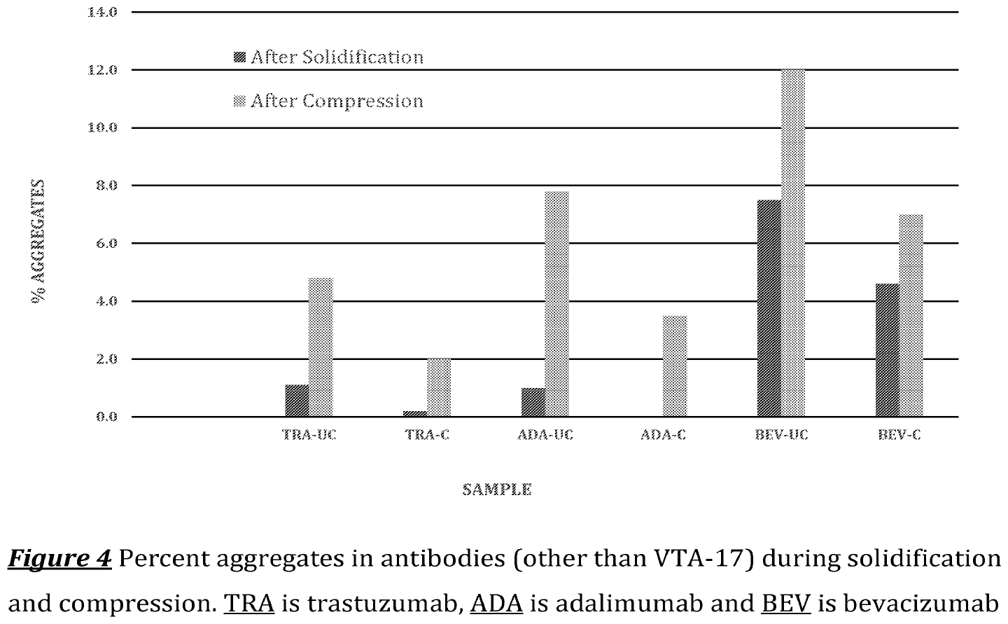

From the results provided by Table 2 & FIG. 2 and Table 3 & FIG. 3, it is evident that CMD and HPBCD play a significant role in protecting the integrity of VTA-17 against aggregation formed during evaporative solidification and compression. Three other monoclonal antibodies were also tested and exhibited similar phenomena where CMD and HPBCD offer unexpected significant protection by suppressing the aggregate growth during solidification and compression. Three antibodies such as trastizumab, adalimumab and bevacizumab were tested after evaporative solidification and compression before and after complexation with CMD and HPBCD. The results were shown in FIG. 4 & table 4.

As obtained, there is no aggregates reported by the supplier for these antibodies. For uncomplexed trastizumab, the aggregates grew to 1.1% after solidification and to 4.8% after compression whereas for complexed trastizumab, the aggregate growth is well controlled to 0.2% after solidification and 2.0% after compression (FIG. 4 & table 4). For uncomplexed adalimumab, the aggregates grew to 1.0% after solidification and to 7.8% after compression whereas for complexed adalimumab, no aggregates detected after solidification and 3.5% of aggregates observed after compression (FIG. 4 & table 4). For uncomplexed bevacizumab, the aggregates grew to 7.5% after solidification and to 12.0% after compression whereas for complexed bevacizumab, the percent aggregates is 4.6% after solidification and 7.0% after compression (FIG. 4 & Table 4). From the data of these three antibodies, it is clear that the complexation with CMD & HPBCD has a significant role in controlling the aggregation. The magnitude of the effect of complexation in the case of trastizumab, adalimumab and bevacizumab is not that great when compared with that of VTA-17. The reason is that these three commercially available antibodies when purchased already existed in a formulated state with lot of stabilizers already present in their solutions. In spite of stabilizers already present in their solutions, the present method had shown additional protection to these three mAbs during solidification and compression.

TABLE 4
Percent aggregates in antibodies (other than
VTA-17) during solidification and
compression. TRA is trastuzumab,
ADA is adalimumab and BEV is bevacizumab

| | | % Aggregates | |
|---|---|---|---|
| | | After | After |
| Sample | | Solidification | Compression |
| Trastuzumab | Uncomplexed | 1.1 | 4.8 |
| | Complexed | 0.2 | 2.0 |
| Adalimumab | Uncomplexed | 1.0 | 7.8 |
| | Complexed | 0.0 | 3.5 |
| Bevacizumab | Uncomplexed | 7.5 | 12.0 |
| | Complexed | 4.6 | 7.0 |

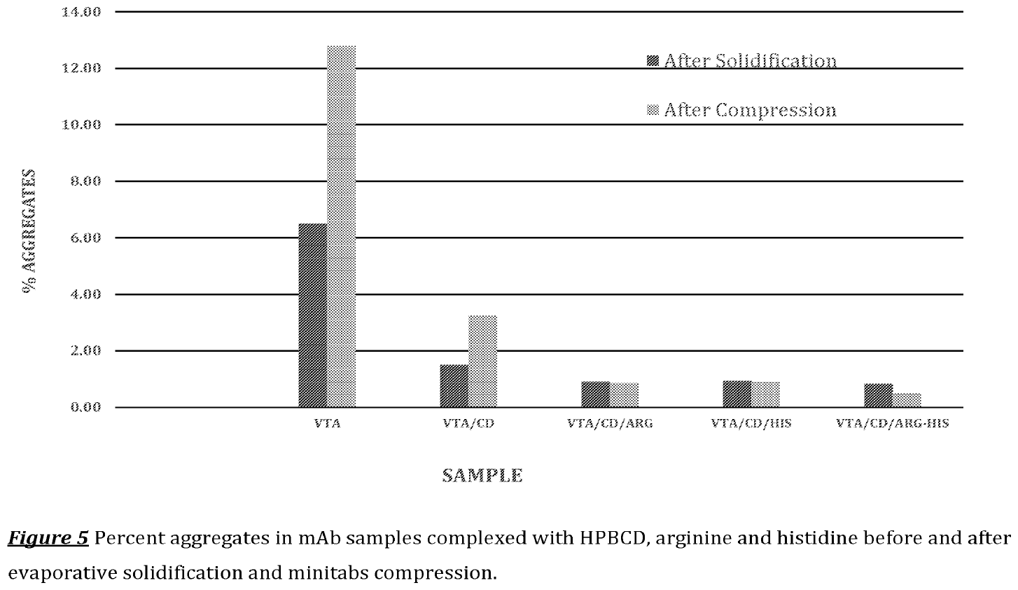

The inventors discovered that basic amino acids such as arginine and histidine in combination with HPBCD also exhibit a role in controlling the aggregate growth of VTA-17 during evaporative solidification and compression. VTA-17 was complexed with HPBCD, arginine HCl and histidine HCl in various combinations as shown in table 5. The resulting solutions after complexation were solidified and the obtained solid powders were compressed to minitabs according to the procedure mentioned earlier. The results were shown in FIG. 5 and table 6. As mentioned before, during evaporative solidification, the aggregates of uncomplexed VTA-17 grew to 6.5%. For the VTA-17 complexed with only HPBCD, the aggregates grew to 1.5%. But for the VTA-17 complexed with combination of HPBCD with arginine or histidine or both, the aggregate growth during solidification was well controlled to less than 1.0% (FIG. 5 & Table 6). In addition, an ELISA assay showed that the potency of the VTA-17 remained unaffected.

A similar trend was observed during the compression. During the compression, the aggregates of uncomplexed VTA-17 grew to 12.8%. The aggregates grew to 3.2% for the VTA-17 complexed with only HPBCD. The aggregates are very well controlled to less than 1.0% during the compression for the VTA-17 complexed with the combination of HPBCD with arginine or histidine or both (See the FIG. 5 and Table 6). In fact, these basic amino acids (arginine HCl and histidine HCl) in combination with HPBCD offered competitive protection to VTA-17 during solidification and compression when compared with that of CMD and HPBCD. The positively charged basic amino acids may align along the protein backbone through electrostatic attractive forces making the antibody unsusceptible to aggregation.

TABLE 5
Amount of ingredients used to make mAb-
HPBCD-Arginine-Histidine complex materials

| | Amount (mg) | | | |
|---|---|---|---|---|
| | | | Arginine | Histidine |
| S. No. | mAb | HPBCD | HCl | HCl |
| 1 | 200 | 0 | 0 | 0 |
| 2 | 200 | 500 | 0 | 0 |
| 3 | 200 | 500 | 200 | 0 |
| 4 | 200 | 500 | 0 | 200 |
| 5 | 200 | 500 | 200 | 200 |

TABLE 6
Percent aggregates in mAb samples complexed with HPBCD, Arginine and
Histidine before and after evaporative solidification and minitabs compression

| | | After | After |
|---|---|---|---|
| Sample | Sample Description | Solidification | Compression |
| VTA-17 | VTA-17 sample by itself | 6.5 | 12.80 |
| VTA-17/CD | VTA-17 complexed with HPBCD | 1.50 | 3.25 |
| VTA-17/CD/ARG | VTA-17 complexed with arginine HCl | 0.91 | 0.85 |
| VTA-17/CD/HIS | VTA-17 complexed with histidine HCl | 0.93 | 0.90 |
| VTA-17/CD/ARG-HIS | VTA-17 complexed with arginine HCl | 0.82 | 0.50 |
| | and Histine HCl | | |

These results have an important implication in designing formulations that require conversion of VTA-17 solution to a flowable powder intended for solid oral dosage forms, like tablets and minitabs. It is important to note that, in general, protein denaturation (due to generation of aggregates) can be initiated by a pressure as low as 1 kbar (Bridgman, P. W., The Coagulation of Albumin by Pressure. Journal of Biological Chemistry 1914, 19, 511-512). Despite the fact that the present formulation of minitabs (containing the mAb) were compressed at considerably higher pressure (3.5 to 10.5 kbar), they exhibited minimal to no aggregate content after undergoing two processes of evaporative solidification and compression substantiating the considerable protection properties of CMD & HPBCD and HPBCD and basic amino acids (arginine and histidine) on the VTA-17 against compression and evaporation.

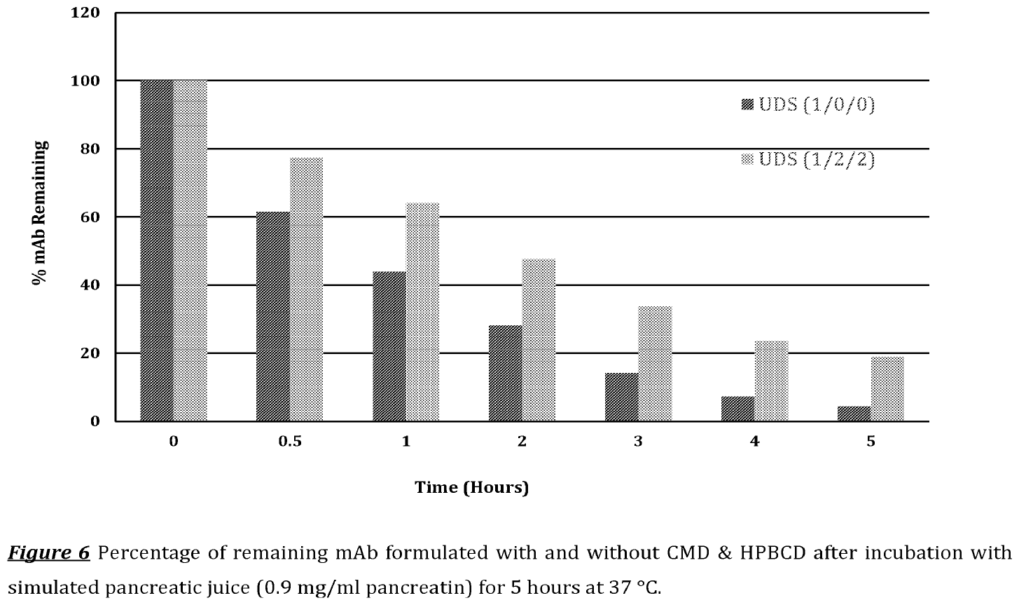

The inventors discovered that CMD and HPBCD together also play a significant role in protecting mAb against proteolytic degradation by digestive enzymes such as pancreatin. Samples of uncomplexed and complexed mAb (in the ratio 1/2/2) were treated with pancreatin (concentration of 0.9 mg/mL) for up to 5 hours at 37° C., and the rate of proteolysis was studied using size exclusion chromatography (SEC). The results shown in FIG. 6, indicate that the use of CMD & HPBCD surprisingly provides protection of mAb from proteolytic degradation.

As shown in FIG. 6, in one hour of pancreatin treatment at 37° C., about 44% of mAb remained intact after proteolysis for uncomplexed mAb, whereas about 64% of complexed mAb (1/2/2) remained intact. Complexed mAb survived significantly better than that of uncomplexed mAb showing significant protection due to CMD and HPBCD against proteolysis. Oral therapeutic drugs usually have a transit time of about 3-4 hours in the small intestine (Davis, S. S.; Hardy, J. G.; Fara, J. W., Transit of pharmaceutical dosage forms through the small intestine. Gut 1986, 27 (8), 886-892). After 4 hours of incubation in pancreatin, 25% from the complexed mAb (1/2/2) survived whereas the mAb from uncomplexed material (1/0/0) showed 7% survival. These observations clearly indicate that CMD and HPBCD formulated mAb significantly resists the proteolysis by pancreatin contributing to a potential successful oral antibody formulation to be delivered in the small intestine and colon of the digestive system. Presented data in FIG. 6 are the average of two determinations (n=2).

While the present disclosure has illustrated by description several embodiments and while the illustrative embodiments have been described in considerable detail, it is not the intention of the applicant to restrict or in any way limit the scope of the appended claims to such detail. Additional advantages and modifications may readily appear to those skilled in the art. Furthermore, features from separate lists can be combined; and features from the examples can be generalized to the whole disclosure.

### EXAMPLES

### Complexation and Evaporative Solidification of Antibodies

The VTA-17 mAb was isolated from transgenic goat milk (U.S. Pat. No. 7,939,317), purified and extracted into an acetate buffer using a validated process per US2017/0121402. The purity of VTA-17 in buffer solution was found to be >99.0% as analyzed by size exclusion chromatography (SEC). From SEC, it was shown that this solution was free from any co-proteins such as β-lactoglobulins, possessed aggregates about 1.0% and was free from any monomer fragments of mAb.

The complexation, evaporative solidification, and compression of VTA-17 was carried out in two separate procedures. In the first process, the solution containing VTA-17 was complexed with CMD and HPBCD at various ratios of mAb/CMD/HPBCD. These complexed VTA-17 materials were solidified and compressed to 2 mm-diameter minitabs. Other monoclonal antibodies such as adalimumab, bevacizumab and trastuzumab were also complexed with CMD and HPBCD in a procedure similar to that of VTA-17. In the second experiment, the solution containing VTA-17 was complexed with HPBCD, arginine HCl and histidine HCl. These complexed VTA-17 materials were solidified and compressed to 2 mm-diameter minitabs.

Complexation of VTA-17 with CMD & HPBCD

The solution containing mAb (VTA-17) complexed with stabilizing excipients: CMD and HPBCD at various (mAb/CMD/HPBCD) w/w/w ratios of (1/0/0), (1/1/1), (1/1.5/1.5), (1/2/2), (1/2/0) and (1/0/2) respectively, shown in Table 1. The complexed solutions were solidified through a vacuum evaporation method using a rotary evaporator. The solvent was gradually evaporated at 40° C. under reduced pressure to obtain a solid material of VTA-17-CMD-HPBCD complex.

The applicability of the complexation method for other monoclonal antibodies than VTA-17 was explored. Monoclonal antibodies such as adalimumab, bevacizumab, and trastuzumab were also complexed with CMD and HPBCD in (mAb/CMD/HPBCD) w/w/w ratio of (1/2/2). The resulting solutions were solidified through vacuum evaporation method using rotary evaporator. By gradual evaporating of the solvent at 40° C. under reduced pressure, a solid material of mAb-CMD-HPBCD complex was obtained.

TABLE 1
Amounts of ingredients used to make mAb-CMD-HPBCD
complex materials

| S. | | Vol | Amount (mg) | | | | |
|---|---|---|---|---|---|---|---|
| No. | Ratio | (mL) | mAb | CMD | HPBCD | Total | Notes |
| 1 | 1/0/0 | 25 | 428 | 0 | 0 | 428 | Uncomplexed mAb |
| 2 | 1/1/1 | 25 | 428 | 428 | 428 | 1283 | mAb complexed with |
| | | | | | | | CMD & HPBCD |
| | | | | | | | in 1:1 ratio |
| 3 | 1/1.5/1.5 | 25 | 428 | 641 | 641 | 1710 | mAb complexed with |
| | | | | | | | CMD & HPBCD |
| | | | | | | | in 1:1.5 ratio |
| 4 | 1/2/2 | 25 | 428 | 855 | 855 | 2138 | mAb complexed with |
| | | | | | | | CMD & HPBCD |
| | | | | | | | in 1:2 ratio |
| 5 | 1/2/0 | 25 | 428 | 855 | 0 | 1283 | mAb complexed with |
| | | | | | | | only CMD |
| | | | | | | | in 1:2 ratio |
| 6 | 1/0/2 | 25 | 428 | 0 | 855 | 1283 | mAb complexed with |
| | | | | | | | only HPBCD |
| | | | | | | | in 1:2 ratio |

Complexation of VTA-17 with HPBCD, Arginine HCl & Histidine HCl

A solution containing VTA-17 was complexed with stabilizing excipients: HPBCD, arginine, and histidine at various combinations as shown in table 2. The complexed solutions were solidified through vacuum evaporation method using a rotary evaporator. The solvent was gradually evaporated at 40° C. under reduced pressure to obtain a solid material of VTA-17 complex.

Although the evaporative solidification process did not involve high temperature, it required a strong vacuum while the solution of complexed VTA-17 was continuously being rotated. The evaporative solidification yielded material with desirable fluidity properties needed for minitab compression. This developed method was found to be superior to a previously implemented spray-drying methods, where a VTA-17 solution (complexed with CMD & HPBCD) was spray-dried resulting in a mAb powder material with a sticky nature, making it inappropriate for tablet compression.

To dry the complexed solutions, a two-stage process of evaporative solidification was carried on a rotary evaporator. Stage 1 involved the loading of previously prepared mAb complexed solution into a round bottom flask fitted on a rotary evaporator setup at low vacuum pull. A simultaneous pulling and filtration took place during this process. Stage 2 involved the actual solidification through vacuum evaporation. The slow evaporation was carried out under vacuum at a reduced pressure at 40° C. with coolant in the cold finger. The resulting material was further dried in a vacuum oven overnight at 40° C. After drying, the materials were triturated to yield the free-flowing powders of VTA-17. The resulting complex materials were analyzed using size exclusion chromatography (SEC) to measure the percent of aggregates generated during the process of evaporative solidification. See Table 6.

### Compression of mAb Minitabs

Compression causes aggregation and denaturation of proteins/antibodies. In this example, mAb was compressed at various compression pressures in the presence/absence of the stabilizers CMD, HPBCD, arginine HCl, and Histidine HCl and the growth of aggregates was studied using SEC. Since 1-2 kbar range was found to be sufficient to initiate aggregation in some proteins, compression pressures of 3.5, 7.0 and 10.5 kbar were chosen.

Minitab powder formulations were prepared by blending dry powder of the mAb with other inactive ingredients recognized by the FDA as GRAS (generally recognized as safe) materials. The inactive ingredients include binders such as microcrystalline cellulose (MCC), hydroxypropyl methyl cellulose (HPMC), glidant such as silicon dioxide, and lubricant such as magnesium stearate. The powder blends were thoroughly mixed except magnesium stearate, which was added and mixed later, just before compression. These blends consisting of mAb complexes were compressed into minitabs at various pressures of 3.5, 7.0 and 10.5 kbar. To determine the percent of aggregates generated during the process of compression, representative samples of minitabs were stirred in phosphate buffer saline (PBS) for one hour to extract mAb from the minitabs into PBS solution. The solutions were filtered using PVDF membrane filters and the filtrates were analyzed using size exclusion chromatography (SEC) to determine the percent of aggregates generated during the process of compression. See Table 3 and Table 6.

Size exclusion chromatography (SEC) is a commonly used analytical method to quantify antibodies, its aggregates, and its degradants. An Acquity UPLC protein BEH SEC column (200 Å, 1.7 μm, 4.6 mm×150 mm) was used to analyze the antibody samples. The mobile phase consists of 10 mM Na₂HPO₄, 1.8 mM KH₂PO₄, 2.7 mM KCl, 400 mM NaCl at pH 6.8. The flow rate of the analysis and the injection volume are 0.4 mL/minute and 2.6 μL respectively. The samples were injected into the HPLC equipped with SEC column and ran for 10 minutes. In the SEC analysis of antibodies, usually aggregates of mAb elute first followed by the antibody and at last the low molecular weight compounds or antibody degradants.

Proteolysis of mAb with Pancreatin

In this example, the resistance towards proteolysis of mAb complexed with CMD & HPBCD was measured. The samples were the dry mAb powders uncomplexed (1/0/0) and complexed with CMD and HPBCD in (1/2/2) ratio. A comparative proteolytic degradation between (1/0/0) and (1/2/2) samples was conducted using pancreatin solution, an intestinal digestive enzyme mixture for up to 5 hours.

Pancreatin solution was prepared by dissolving 12.5 g of NaHCO₃, 6 g of dehydrated bile extract, and 0.9 g of pancreatin in 1 liter of deionized (DI) water. The pH was adjusted to 6.8 with 0.1 N HCl. The pancreatin comprises several digestive enzymes including trypsin, chymotrypsin, caboxypeptidase, pipase, and amylase. Pancreatin solution was added to the solution containing mAb and the reaction continued for 5 hours while aliquots were collected at various time intervals. A proteolysis quencher (a combination of Pefabloc SC Plus at a concentration of 9 mg/mL and Papstatin A at a concentration of 3 mg/mL) was added to the collected samples immediately prior to samples being analyzed using SEC (Acquity UPLC protein BEH SEC column (200 Å, 1.7 μm, 4.6 mm×150 mm)) for the mAb and its degradants. It was found that after two hours, at least 45% of the mAb had not been digested by the pancreatin. Proteolysis resistance is the remaining activity of the mAb after two hours of exposure to the pancreatin solution.

TABLE 7
Percentage of remaining mAb formulated
with and without CMD & HPBCD
after incubation with simulated pancreatic juice
(0.9 mg/ml pancreatin) for 5 hours at 37° C.

| Time | mAb (1/0/0), | mAb (1/2/2), |
|---|---|---|
| (hours) | Uncomplexed | Complexed |
| 0 | 100 | 100 |
| 0.5 | 61.5 | 77.3 |
| 1 | 43.9 | 64.0 |
| 2 | 28.1 | 47.7 |
| 3 | 14.2 | 33.7 |
| 4 | 7.3 | 23.6 |
| 5 | 4.4 | 18.9 |

1. A powder comprising one or more monoclonal antibody, one or more cyclodextrin, and a compound selected from carboxymethyl dextran (CMD), one or more basic amino acid, or both.

2. The powder of claim 1, wherein the cyclodextrin comprises hydroxy propyl beta cyclodextrin (HPBCD).

3. The powder of claim 1, comprising about 20% to about 40% of one or more monoclonal antibody, about 35% to about 70% of one or more cyclodextrin, and about 35% to about 70% CMD.

4. The powder of claim 1, comprising about 20% to about 40% of one or more monoclonal antibody, about 45% to about 70% of one or more cyclodextrin, and about 15% to about 25% of one or more basic amino acid.

5. The powder of any of claim 1, wherein the monoclonal antibody is selected from VTA-17, trastuzumab, adalimumab, bevacizumab, or combinations thereof.

6. The powder of any of claim 1, wherein the one or more basic amino acid comprises an amino acid selected from arginine, histidine, or both.

7. A compressed shape, wherein the shape is formed by compressing the powder of claim 1 at a pressure of about 3.5 to about 10.5 kbar.

8. The compressed shape of claim 7, wherein the aggregates are about 1.5% or less.

9. The compressed shape of claim 7, wherein the proteolysis resistance is 45%.

10. The compressed shape of claim 7, wherein the shape is in the form of 2 mm-diameter minitabs.

11. A method of forming a powder comprising the steps of:
providing one or more monoclonal antibody, one or more cyclodextrin, and a compound selected from carboxymethyl dextran (CMD), one or more basic amino acid, or both;
forming a solution comprising the monoclonal antibody, cyclodextrin, CMD, and amino acid; and
drying the solution.

12. The method of claim 11, wherein the cyclodextrin comprises hydroxy propyl beta cyclodextrin (HPBCD).

13. The method of claim 11, comprising about 20% to about 40% of one or more monoclonal antibody, about 35% to about 70% of one or more cyclodextrin, and about 35% to about 70% CMD.

14. The method of claim 11, comprising about 20% to about 40% of one or more monoclonal antibody, about 45% to about 70% of one or more cyclodextrin, and about 15% to about 25% of one or more basic amino acid.

15. The method of claim 11, wherein the monoclonal antibody is selected from VTA-17, trastuzumab, adalimumab, bevacizumab, or combinations thereof.

16. The method of any of claim 11, wherein the one or more basic amino acid comprises an amino acid selected from arginine, histidine, or both.

17. A method of forming a compressed shape comprising the steps of:
providing the powder of claim 11; and
compressing the powder at a pressure of about 3.5 to about 10.5 kbar.

18. The method of claim 17, wherein the aggregates is about 1.5% or less.

19. The method of claim 17, wherein the proteolysis resistance is 45%.

20. The method of claim 17, wherein the shape is in the form of 2 mm-diameter minitabs.
